(19) United States
(12) Patent Application Publication
(10) Pub. No.: US 2020/0297670 A1
(43) Pub. Date: Sep. 24, 2020

(54) Capsaicin Sequential Dosing Method for Treatment of Morton's Neuroma Pain

(71) Applicant: Centrexion Therapeutics Corporation, Boston, MA (US)

(72) Inventors: James N. Campbell, Baltimore, MD (US); Peter D. Hanson, Prides Crossing, MA (US); Randall Stevens, Rockport, MA (US)

(21) Appl. No.: 16/751,844

(22) Filed: Jan. 24, 2020

Related U.S. Application Data
(63) Continuation of application No. 16/070,889, filed on Jul. 18, 2018, now abandoned.
(60) Provisional application No. 62/281,877, filed on Jan. 22, 2016.

(51) Int. Cl. A61K 31/165; A61P 29/02; A61K 9/00; A61K 47/10

(52) U.S. Cl. CPC A61K 31/165; A61P 29/02; A61K 31/245; A61K 47/10; A61K 9/0019

(57) ABSTRACT

A method of ameliorating pain for a duration of at least 3 months due to an intermetatarsal neuroma in a patient, comprising administering by injection into or adjacent to the patient's intermetatarsal space having an intermetatarsal neuroma at least a first dose of capsaicin and a second dose of capsaicin, no sooner than one month after the first dose, to ameliorate pain due to the intermetatarsal neuroma.

### CROSS REFERENCE TO RELATED APPLICATIONS

This application claims the benefit of and priority to U.S. Provisional Patent Application Ser. No. 62/281,877, filed Jan. 22, 2016, the contents of which are hereby incorporated by reference.

### FIELD OF THE INVENTION

The invention provides methods and compositions for sequential dosing of capsaicin to treat pain due to an intermetatarsal neuroma in a patient.

### BACKGROUND

Intermetatarsal neuroma is a painful condition in the web space of the foot typically caused at least in part by compression of the distal common digital nerve in the intermetatarsal space. The condition is most common in the third intermetatarsal space, followed in incidence by involvement of the second intermetatarsal space. In some instances, a patient may suffer from an intermetatarsal neuroma in both the second intermetatarsal space and the third intermetatarsal space. Patients suffering from an intermetatarsal neuroma may experience pain upon standing and walking, which can be associated with numbness and/or paresthesias extending to the toes.

Existing therapies do not meet the needs for all patients and/or have significant drawbacks. For example, one approach sometimes used to achieve relief from pain of an intermetatarsal neuroma is surgical excision of the common digital nerve, which may involve resection of the involved nerve, decompression surgery, or cryogenic neuroablation of the neuroma. However, surgical excision of the common digital nerve leads to permanent loss of sensation in the toes and may be associated with additional complications. Non-surgical options for relieving the pain of an intermetatarsal neuroma include changes in footwear (e.g., a wide toe box), use of metatarsal pads, use of orthotics, injection of steroids or sclerosing agents (e.g., phenol) into the area of the intermetatarsal neuroma, and/or administration of an oral analgesic. However, these non-surgical options are not effective in sufficiently reducing pain due to the intermetatarsal neuroma for all patients.

Accordingly, the need exists for new procedures for treating pain due to an intermetatarsal neuroma. The present invention addresses this need and provides other related advantages.

### SUMMARY

The invention provides methods and compositions for sequential dosing of capsaicin to treat pain due to an intermetatarsal neuroma in a patient. The methods desirably provide relief from pain due to the intermetatarsal neuroma for an extended duration, such as at least about 3 months, 6 months, 9 months, or 1 year. The methods generally involve administering at least two doses of capsaicin to the patient by injection into the patient's intermetatarsal space having an intermetatarsal neuroma. Preferably, the instrument used to perform the injection of capsaicin does not penetrate into the intermetatarsal neuroma, but rather distributes capsaicin to tissue in proximity to the intermetatarsal neuroma. After administering the first dose of capsaicin, the second and any subsequent dose of capsaicin is desirably administered before the patient begins to experience any significant pain due to the intermetatarsal neuroma. In a preferred embodiment, a single dose of capsaicin (e.g., a 200 μg dose of capsaicin) ameliorates pain due the intermetatarsal neuroma for a duration of at least 2 months, 3 months, 4 months, or even longer (e.g., at least one year). Various aspects and embodiments of the invention are described in further detail below.

One aspect of the invention provides a method of ameliorating pain for a duration of at least 6 months due to an intermetatarsal neuroma in a patient. The method comprises administering by injection into the patient's intermetatarsal space having an intermetatarsal neuroma at least a first dose of capsaicin and a second dose of capsaicin to ameliorate pain due to the intermetatarsal neuroma for a duration of at least 6 months, wherein the method is characterized by: (a) the first dose of capsaicin is in an amount ranging from about 150 μg to about 250 μg of capsaicin; (b) the second dose of capsaicin is in an amount ranging from about 150 μg to about 250 μg of capsaicin; (c) the second dose of capsaicin is administered no sooner than 3 months after administration of the first dose of capsaicin; and (d) if any additional dose of capsaicin is administered by injection into the patient's intermetatarsal space having an intermetatarsal neuroma, any such additional dose is in an amount ranging from about 150 μg to about 250 μg of capsaicin and any said additional dose is administered no sooner than 3 months after administration of the prior dose of capsaicin administered by injection into the patient's intermetatarsal space having an intermetatarsal neuroma. In a preferred embodiment, the first dose of capsaicin is about 200 μg of capsaicin, and the second dose of capsaicin is about 200 μg of capsaicin which is administered from 3 months to 5 months after administering the first dose of capsaicin to the patient. The capsaicin is preferably administered as an injectable solution containing water and a poly(ethylene glycol), wherein the injectable solution has a volume of about 2 mL.

Another aspect of the invention provides a method of ameliorating pain for a duration of at least 3 months due to an intermetatarsal neuroma in a patient. The method comprises administering by injection into the patient's intermetatarsal space having an intermetatarsal neuroma at least a first dose of capsaicin and a second dose of capsaicin to ameliorate pain due to the intermetatarsal neuroma for a duration of at least 3 months, wherein the method is characterized by: (a) the first dose of capsaicin is in an amount ranging from about 100 μg to about 1,000 μg of capsaicin; (b) the second dose of capsaicin is in an amount ranging from about 100 μg to about 1,000 μg of capsaicin; (c) the second dose of capsaicin is administered no sooner than 1 month after administration of the first dose of capsaicin; and (d) if any additional dose of capsaicin is administered by injection into the patient's intermetatarsal space having an intermetatarsal neuroma, any such additional dose is in an amount ranging from about 100 μg to about 1,000 μg of capsaicin and any said additional dose is administered no sooner than 1 month after administration of the prior dose of capsaicin administered by injection into the patient's intermetatarsal space having an intermetatarsal neuroma. In a preferred embodiment, the first dose of capsaicin is about 200 μg of capsaicin, and the second dose of capsaicin is about 200 μg of capsaicin which is administered from 3 months to 5 months after administering the first dose of capsaicin to the patient. The capsaicin is preferably administered as an injectable solution containing water and a poly(ethylene glycol), wherein the injectable solution has a volume of about 2 mL.

### DETAILED DESCRIPTION

The invention provides methods and compositions for sequential dosing of capsaicin to treat pain due to an intermetatarsal neuroma in a patient. The methods generally involve administering at least two doses of capsaicin to the patient by injection into the patient's intermetatarsal space having an intermetatarsal neuroma. Preferably, the instrument used to perform the injection of capsaicin does not penetrate into the intermetatarsal neuroma, but rather distributes capsaicin to tissue in proximity to the intermetatarsal neuroma. After administering the first dose of capsaicin, the second and any subsequent dose of capsaicin is desirably administered before the patient begins to experience any significant pain due to the intermetatarsal neuroma. In a preferred embodiment, a single dose of capsaicin (e.g., a 200 μg dose of capsaicin) ameliorates pain due the intermetatarsal neuroma for a duration of at least 2 months, 3 months, 4 months, or even longer (e.g., at least one year). The magnitude of pain experienced by a patient may be evaluated using procedures described in the literature, such as the Numeric Pain Rating Scale (NPRS), where pain is characterized by the patient on a scale of zero to ten (with zero being “no pain”, and ten being “worst possible pain”). The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The phrase “Injection Pain Scale” refers to a measure of pain experienced by a patient upon administration of capsaicin by injection, where the extent of pain experienced by the patient is rated by the patient as one of the following: (i) none, (ii) mild pain, (iii) moderate pain, or (iv) intense pain.

Compounds of the disclosure may contain a C—C double bond and, therefore, exist as geometric isomers. Individual geometric isomers of compounds of the present invention can be prepared synthetically from commercially available starting materials that contain a single geometric isomer in high purity and/or through separating a mixture of geometric isomers using chromatographic procedures known in the art. Substituents around a carbon-carbon double bond are designated as being in the “Z” or “E” configuration wherein the terms “Z” and “E” are used in accordance with IUPAC standards. Substituents around a carbon-carbon double bond alternatively can be referred to as “cis” or “trans,” where “cis” represents substituents on the same side of the double bond and “trans” represents substituents on opposite sides of the double bond.

The compounds may be in amorphic or crystalline form, and the invention encompasses all such amorphic and crystalline forms.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Therapeutic Applications

One aspect of the invention provides methods for sequential dosing of capsaicin to treat pain due to an intermetatarsal neuroma in a patient. The methods desirably provide relief from pain due to the intermetatarsal neuroma for an extended duration, such as at least about 3 months, 6 months, 9 months, or 1 year. The methods generally involve administering at least two doses of capsaicin to the patient by injection into the patient's intermetatarsal space having an intermetatarsal neuroma. Preferably, the instrument used to perform the injection of capsaicin does not penetrate into the intermetatarsal neuroma, but rather distributes capsaicin to tissue in proximity to the intermetatarsal neuroma. After administering the first dose of capsaicin, the second and any subsequent dose of capsaicin is desirably administered before the patient begins to experience any significant pain due to the intermetatarsal neuroma. In a preferred embodiment, a single dose of capsaicin ameliorates pain due the intermetatarsal neuroma for a duration of at least 2 months, 3 months, 4 months, or even longer (e.g., at least one year). Various aspects and embodiments of the methods are described below.

### First Method

One aspect of the invention provides a method of ameliorating pain for a duration of at least 6 months due to an intermetatarsal neuroma in a patient. The method comprises administering by injection into the patient's intermetatarsal space having an intermetatarsal neuroma at least a first dose of capsaicin and a second dose of capsaicin to ameliorate pain due to the intermetatarsal neuroma for a duration of at least 6 months, wherein the method is characterized by: (a) the first dose of capsaicin is in an amount ranging from about 150 μg to about 250 μg of capsaicin; (b) the second dose of capsaicin is in an amount ranging from about 150 μg to about 250 μg of capsaicin; (c) the second dose of capsaicin is administered no sooner than 3 months after administration of the first dose of capsaicin; and (d) if any additional dose of capsaicin is administered by injection into the patient's intermetatarsal space having an intermetatarsal neuroma, any such additional dose is in an amount ranging from about 150 μg to about 250 μg of capsaicin and any said additional dose is administered no sooner than 3 months after administration of the prior dose of capsaicin administered by injection into the patient's intermetatarsal space having an intermetatarsal neuroma.

The method may be further characterized according to the dose of capsaicin administered to the patient. For example, in certain embodiments, the first dose of capsaicin is in an amount ranging from about 175 μg to about 225 μg of capsaicin. In certain embodiments, the first dose of capsaicin is about 200 μg of capsaicin. In certain embodiments, the second dose of capsaicin is in an amount ranging from about 175 μg to about 225 μg of capsaicin. In certain embodiments, the second dose of capsaicin is about 200 μg of capsaicin. In certain embodiments, any additional dose of capsaicin is in an amount ranging from about 175 μg to about 225 μg of capsaicin. In certain embodiments, the any additional dose of capsaicin is about 200 μg of capsaicin.

### Second Method

Another aspect of the invention provides a method of ameliorating pain for a duration of at least 3 months due to an intermetatarsal neuroma in a patient. The method comprises administering by injection into the patient's intermetatarsal space having an intermetatarsal neuroma at least a first dose of capsaicin and a second dose of capsaicin to ameliorate pain due to the intermetatarsal neuroma for a duration of at least 3 months, wherein the method is characterized by: (a) the first dose of capsaicin is in an amount ranging from about 100 μg to about 1,000 μg of capsaicin; (b) the second dose of capsaicin is in an amount ranging from about 100 μg to about 1,000 μg of capsaicin; (c) the second dose of capsaicin is administered no sooner than 1 month after administration of the first dose of capsaicin; and (d) if any additional dose of capsaicin is administered by injection into the patient's intermetatarsal space having an intermetatarsal neuroma, any such additional dose is in an amount ranging from about 100 μg to about 1,000 μg of capsaicin and any said additional dose is administered no sooner than 1 month after administration of the prior dose of capsaicin administered by injection into the patient's intermetatarsal space having an intermetatarsal neuroma.

The method may be further characterized according to the dose of capsaicin administered to the patient. For example, in certain embodiments, the first dose of capsaicin is in an amount ranging from about 100 μg to about 300 μg of capsaicin. In certain embodiments, the first dose of capsaicin is in an amount ranging from about 150 μg to about 250 μg of capsaicin. In certain embodiments, the first dose of capsaicin is about 200 μg of capsaicin. In certain embodiments, the second dose of capsaicin is in an amount ranging from about 100 μg to about 300 μg of capsaicin. In certain embodiments, the second dose of capsaicin is in an amount ranging from about 150 μg to about 250 μg of capsaicin. In certain embodiments, the second dose of capsaicin is about 200 μg of capsaicin. In certain embodiments, the any additional dose of capsaicin is in an amount ranging from about 100 μg to about 300 μg of capsaicin. In certain embodiments, the any additional dose of capsaicin is in an amount ranging from about 150 μg to about 250 μg of capsaicin. In certain embodiments, the any additional dose of capsaicin is about 200 μg of capsaicin.

The method may be further characterized according to the duration over which pain is ameliorated. For example, in certain embodiments, the pain is ameliorated for a duration of at least 4 months. In certain embodiments, the pain is ameliorated for a duration of at least 5 months. In certain embodiments, the pain is ameliorated for a duration of at least 6 months.

The method may be further characterized according to the time at which the second dose of capsaicin is administered to the patient. For example, in certain embodiments, the second dose of capsaicin is administered no sooner than 2 months after administration of the first dose of capsaicin. In certain embodiments, the second dose of capsaicin is administered at a time that is in the range of 1 month to 3 months after administration of the first dose of capsaicin. In certain embodiments, the second dose of capsaicin is administered at a time that is in the range of 2 months to 4 months after administration of the first dose of capsaicin.

### Third Method

Another aspect of the invention provides a method of ameliorating pain for a duration of at least 3 months due to an intermetatarsal neuroma in a patient. The method comprises administering by injection into the patient's intermetatarsal space having an intermetatarsal neuroma at least a first dose of capsaicin and a second dose of capsaicin to ameliorate pain due to the intermetatarsal neuroma for a duration of at least 3 months, wherein the method is optionally characterized by one or more of: (a) the first dose of capsaicin is in an amount ranging from about 100 μg to about 1,000 μg of capsaicin; (b) the second dose of capsaicin is in an amount ranging from about 100 μg to about 1,000 μg of capsaicin; (c) the second dose of capsaicin is administered no sooner than 1 week after administration of the first dose of capsaicin; and (d) if any additional dose of capsaicin is administered by injection into the patient's intermetatarsal space having an intermetatarsal neuroma, any such additional dose is in an amount ranging from about 100 μg to about 1,000 μg of capsaicin and any said additional dose is administered no sooner than 1 week after administration of the prior dose of capsaicin administered by injection into the patient's intermetatarsal space having an intermetatarsal neuroma.

### Exemplary Features of the First, Second, and Third Methods

The above methods may be further characterized by additional features, such as the time at which the second dose of capsaicin is administered, time at which a dose of capsaicin subsequent to the second dose of capsaicin is administered, total number of doses of capsaicin administered to the patient, the duration of pain relief provided by the method, and the like. Some of these features are recited above. A more thorough description of such features is provided below. The invention embraces all permutations and combinations of these features.

Time at which Second Dose of Capsaicin is Administered

The methods may be further characterized according to the time at which the second dose of capsaicin is administered to the patient. For example, in certain embodiments, the second dose of capsaicin is administered no sooner than 4 months after administration of the first dose of capsaicin. In certain embodiments, the second dose of capsaicin is administered no sooner than 5 months after administration of the first dose of capsaicin. In certain embodiments, the second dose of capsaicin is administered no sooner than 6 months after administration of the first dose of capsaicin. In certain embodiments, the second dose of capsaicin is administered no sooner than 7 months after administration of the first dose of capsaicin. In certain embodiments, the second dose of capsaicin is administered no sooner than 8 months after administration of the first dose of capsaicin. In certain embodiments, the second dose of capsaicin is administered no sooner than 9 months after administration of the first dose of capsaicin. In certain embodiments, the second dose of capsaicin is administered no sooner than 10 months after administration of the first dose of capsaicin. In certain embodiments, the second dose of capsaicin is administered no sooner than 11 months after administration of the first dose of capsaicin. In certain embodiments, the second dose of capsaicin is administered no sooner than 12 months after administration of the first dose of capsaicin.

In certain embodiments, the second dose of capsaicin is administered at a time that is in the range of 3 months to 5 months after administration of the first dose of capsaicin. In certain embodiments, the second dose of capsaicin is administered at a time that is in the range of 4 months to 6 months after administration of the first dose of capsaicin. In certain embodiments, the second dose of capsaicin is administered at a time that is in the range of 5 months to 7 months after administration of the first dose of capsaicin. In certain embodiments, the second dose of capsaicin is administered at a time that is in the range of 6 months to 8 months after administration of the first dose of capsaicin. In certain embodiments, the second dose of capsaicin is administered at a time that is in the range of 7 months to 9 months after administration of the first dose of capsaicin. In certain embodiments, the second dose of capsaicin is administered at a time that is in the range of 8 months to 10 months after administration of the first dose of capsaicin. In certain embodiments, the second dose of capsaicin is administered at a time that is in the range of 9 months to 11 months after administration of the first dose of capsaicin. In certain embodiments, the second dose of capsaicin is administered at a time that is in the range of 10 months to 12 months after administration of the first dose of capsaicin. In certain embodiments, the second dose of capsaicin is administered at a time that is in the range of 11 months to 13 months after administration of the first dose of capsaicin.

In certain other embodiments, the second dose of capsaicin is administered at about 4 months after administration of the first dose of capsaicin. In certain other embodiments, the second dose of capsaicin is administered at about 5 months after administration of the first dose of capsaicin. In certain other embodiments, the second dose of capsaicin is administered at about 6 months after administration of the first dose of capsaicin. In certain other embodiments, the second dose of capsaicin is administered at about 7 months after administration of the first dose of capsaicin. In certain other embodiments, the second dose of capsaicin is administered at about 8 months after administration of the first dose of capsaicin. In certain other embodiments, the second dose of capsaicin is administered at about 9 months after administration of the first dose of capsaicin. In certain other embodiments, the second dose of capsaicin is administered at about 10 months after administration of the first dose of capsaicin. In certain other embodiments, the second dose of capsaicin is administered at about 11 months after administration of the first dose of capsaicin. In certain other embodiments, the second dose of capsaicin is administered at about 12 months after administration of the first dose of capsaicin.

Patients that have a condition featuring relatively slower nerve growth in the area of the intermetatarsal neuroma (e.g., patients suffering from diabetes mellitus, a toxic neuropathy, or other condition that slows the rate of nerve growth) may benefit from methods where a relatively longer duration of time elapses between administration of the first and second dose of capsaicin. For example, in certain embodiments, the method is characterized by the patient having a condition featuring relatively slower nerve growth in the area of the intermetatarsal neuroma (e.g., patients suffering from diabetes mellitus, a toxic neuropathy, or other condition that slows the rate of nerve growth) and the second dose of capsaicin is administered about 6, 7, 8, 9, 10, 11, or 12 months or longer after administration of the first dose of capsaicin.

Time at which a Dose of Capsaicin Subsequent to the Second Dose of Capsaicin is Administered

The methods may be further characterized according to the time at which a dose of capsaicin subsequent to the second dose of capsaicin is administered to the patient. For example, in certain embodiments, any dose of capsaicin subsequent to the second dose of capsaicin is administered no sooner than 4 months after administration of the prior dose of capsaicin. In certain embodiments, any dose of capsaicin subsequent to the second dose of capsaicin is administered no sooner than 5 months after administration of the prior dose of capsaicin. In certain embodiments, any dose of capsaicin subsequent to the second dose of capsaicin is administered no sooner than 6 months after administration of the prior dose of capsaicin. In certain embodiments, any dose of capsaicin subsequent to the second dose of capsaicin is administered no sooner than 7, 8, 9, 10, 11, or 12 months after administration of the prior dose of capsaicin.

In certain embodiments, any dose of capsaicin subsequent to the second dose of capsaicin is administered at a time that is in the range of 3 months to 5 months after administration of the prior dose of capsaicin. In certain embodiments, any dose of capsaicin subsequent to the second dose of capsaicin is administered at a time that is in the range of 4 months to 6 months after administration of the prior dose of capsaicin. In certain embodiments, any dose of capsaicin subsequent to the second dose of capsaicin is administered at a time that is in the range of 5 months to 7 months after administration of the prior dose of capsaicin. In certain embodiments, any dose of capsaicin subsequent to the second dose of capsaicin is administered at a time that is in the range of 6 months to 8 months after administration of the prior dose of capsaicin. In certain embodiments, any dose of capsaicin subsequent to the second dose of capsaicin is administered at a time that is in the range of 7 months to 9 months after administration of the prior dose of capsaicin. In certain embodiments, any dose of capsaicin subsequent to the second dose of capsaicin is administered at a time that is in the range of 8 months to 10 months after administration of the prior dose of capsaicin. In certain embodiments, any dose of capsaicin subsequent to the second dose of capsaicin is administered at a time that is in the range of 9 months to 11 months after administration of the prior dose of capsaicin.

In certain other embodiments, any dose of capsaicin subsequent to the second dose of capsaicin is administered about 4 months after administration of the prior dose of capsaicin. In certain other embodiments, any dose of capsaicin subsequent to the second dose of capsaicin is administered about 5 months after administration of the prior dose of capsaicin. In certain other embodiments, any dose of capsaicin subsequent to the second dose of capsaicin is administered about 6 months after administration of the prior dose of capsaicin. In certain other embodiments, any dose of capsaicin subsequent to the second dose of capsaicin is administered about 7 months after administration of the prior dose of capsaicin. In certain other embodiments, any dose of capsaicin subsequent to the second dose of capsaicin is administered about 8 months after administration of the prior dose of capsaicin. In certain other embodiments, any dose of capsaicin subsequent to the second dose of capsaicin is administered about 9 months after administration of the prior dose of capsaicin. In certain other embodiments, any dose of capsaicin subsequent to the second dose of capsaicin is administered about 10 months after administration of the prior dose of capsaicin. In certain other embodiments, any dose of capsaicin subsequent to the second dose of capsaicin is administered about 11 months after administration of the prior dose of capsaicin. In certain other embodiments, any dose of capsaicin subsequent to the second dose of capsaicin is administered about 12 months after administration of the prior dose of capsaicin.

Patients that have a condition featuring relatively slower nerve growth in the area of the intermetatarsal neuroma (e.g., patients suffering from diabetes mellitus, a toxic neuropathy, or other condition that slows the rate of nerve growth) may benefit from methods where a relatively longer duration of time elapses between administration of consecutive doses of capsaicin. For example, in certain embodiments, the method is characterized by the patient having a condition featuring relatively slower nerve growth in the area of the intermetatarsal neuroma (e.g., patients suffering from diabetes mellitus, a toxic neuropathy, or other condition that slows the rate of nerve growth) and any dose of capsaicin subsequent to the second dose of capsaicin is administered about 6, 7, 8, 9, 10, 11, or 12 months or longer after administration of the prior dose of capsaicin.

### Total Number of Doses of Capsaicin

The methods may be further characterized according to the total number of doses of capsaicin administered to the patient. For example, in certain embodiments, over a duration of 1 year, the patient receives no more than four doses of capsaicin by injection into the patient's intermetatarsal space having an intermetatarsal neuroma. In certain embodiments, over a duration of 1 year, the patient receives no more than three doses of capsaicin by injection into the patient's intermetatarsal space having an intermetatarsal neuroma. In certain embodiments, over a duration of 1 year, the patient receives no more than two doses of capsaicin by injection into the patient's intermetatarsal space having an intermetatarsal neuroma.

The methods may also be characterized according to the number of additional doses of capsaicin administered to the patient subsequent to the second dose of capsaicin. For example, in certain embodiments, the patient receives at least 1, 2, 3, 4, 5, 6, 7, 8, 9, 10, 11, 12, 15, 20, 25, or 30 additional doses of capsaicin beyond the second dose of capsaicin. In certain embodiments, the patient receives from 1 to 3, 1 to 5, 1 to 10, 5 to 10, 5 to 15, 10 to 15, 10 to 20, 15 to 20, or 15 to 25 additional doses of capsaicin subsequent to the second dose of capsaicin. In certain preferred embodiments, the patient receives at least two additional doses of capsaicin subsequent to the second dose of capsaicin. In yet other embodiments, the patient receives at least four additional doses of capsaicin subsequent to the second dose of capsaicin. In yet other embodiments, the patient receives at least six additional doses of capsaicin subsequent to the second dose of capsaicin.

Patients may continue to receive capsaicin by injection to ameliorate pain due an intermetatarsal neuroma for many months and even multiple years so long as medically prudent, such as the pain relief therapy is well tolerated and sufficiently ameliorates the pain.

### Duration of Pain Relief

The methods may be further characterized according to the duration over which pain due to the intermetatarsal neuroma is ameliorated. For example, in certain embodiments, the pain is ameliorated for a duration of at least 7 months. In certain embodiments, the pain is ameliorated for a duration of at least 8 months. In certain embodiments, the pain is ameliorated for a duration of at least 9 months. In certain embodiments, the pain is ameliorated for a duration of at least 10 months. In certain embodiments, the pain is ameliorated for a duration of at least 11 months. In certain embodiments, the pain is ameliorated for a duration of at least 12 months. In yet other embodiments, the pain is ameliorated for a duration of from about 3 months to about 6 months, from about 3 months to about 9 months, from about 3 months to about 12 months, from about 3 months to about 24 months, from about 6 months to about 12 months, from about 6 months to about 24 months, or from about 12 months to about 24 months.

### Capsaicin

Capsaicin has the chemical name N-[(4-hydroxy-3-methoxyphenyl)methyl]-8-methylnon-6-enamide, and due to the presence of a C—C double bond can exist as a mixture of cis and trans isomers. The methods may be further characterized according to the isomeric purity of the capsaicin administered to the patient. For example, in certain embodiments, the capsaicin is a mixture of cis-capsaicin and trans-capsaicin that contains at least 95% by weight trans-capsaicin. In certain embodiments, the capsaicin is a mixture of cis-capsaicin and trans-capsaicin that contains at least 98% by weight trans-capsaicin. In certain embodiments, the capsaicin is a mixture of cis-capsaicin and trans-capsaicin that contains at least 99% by weight trans-capsaicin.

### Formulations for Injection

The methods may be further characterized according to the formulation used to administer capsaicin to the patient. For example, in certain embodiments, the capsaicin is administered in the form of a liquid, injectable pharmaceutical formulation comprising a pharmaceutically acceptable carrier for injection into a patient. In certain embodiments, the liquid, injectable pharmaceutical formulation comprises water, capsaicin, and a poly(ethylene glycol). In certain other embodiments, the liquid, injectable pharmaceutical formulation consists essentially of water, capsaicin, and a poly(ethylene glycol).

The formulations may be further characterized according to the poly(ethylene glycol) used in the formulation, such as where the poly(ethylene glycol) has a number-average molecular weight of about 250 g/mol to about 350 g/mol. In certain embodiments, the poly(ethylene glycol) has a number-average molecular weight of about 300 g/mol.

The formulations may be further characterized according to the amount of poly(ethylene glycol) used in the formulation, such as where the poly(ethylene glycol) is present in an amount ranging from about 25% to about 35% by weight of the pharmaceutical formulation. In certain embodiments, the poly(ethylene glycol) is present in an amount of about 30% by weight of the pharmaceutical formulation.

### Volume of Unit Dose Liquid Formulation Administered to the Patient

The methods may be further characterized according to amount of the formulation administered to the patient per injection. For example, in certain embodiments, the first dose of capsaicin, the second dose of capsaicin, and the any additional dose of capsaicin are individually a liquid, injectable pharmaceutical formulation having a volume in the range of about 1 to 3 mL. In other embodiments, the first dose of capsaicin, the second dose of capsaicin, and the any additional dose of capsaicin are individually a liquid, injectable pharmaceutical formulation having a volume of about 2 mL.

In certain other embodiments, the volume administered may be less, such as when administering to a pediatric patient. In certain embodiments, the first dose of capsaicin, the second dose of capsaicin, and the any additional dose of capsaicin are individually a liquid, injectable pharmaceutical formulation having a volume in the range of about 0.25 to 2 mL, 0.25 to 1 mL, 0.5 to 1 mL, or 0.5 to 1.5 mL.

### Injection Procedure

The methods may be further characterized according to identity of tissue into which the capsaicin is injected. For example, in certain embodiments, any dose of capsaicin is injected into tissue adjacent to the intermetatarsal neuroma, whereby the medical instrument performing the injection does not penetrate into the intermetatarsal neuroma. It is understood that the injected capsaicin may diffuse through tissue adjacent to the intermetatarsal neuroma in order to reach the intermetatarsal neuroma. Ultrasound imaging may be used by medical personnel performing the injection to help guide the medical instrument (e.g., a syringe) used to administer the formulation containing capsaicin; this procedure helps ensure that the medical instrument performing the injection does not penetrate into the intermetatarsal neuroma but rather delivers capsaicin to tissue adjacent to the intermetatarsal neuroma so that the capsaicin may contact the intermetatarsal neuroma by diffusing through tissue adjacent to the intermetatarsal neuroma.

### Avoidance of Heat

The methods may be further characterized according to activities to be avoided by the patient after being administered the capsaicin. For example, in certain embodiments, the patient does not expose area receiving a capsaicin dose to heat for a duration of at least 24 hours after administration of the capsaicin dose.

### Cooling Tissue Adjacent to Intermetatarsal Neuroma

The methods may be further characterized according to steps taken to minimize pain experienced by the patient due to injection of the capsaicin, such as a step taken to reduce the temperature of tissue adjacent to the intermetatarsal neuroma before and/or after administration of the capsaicin. In certain embodiments, the method further comprises cooling tissue adjacent to the intermetatarsal neuroma before administering capsaicin. In certain embodiments, the method further comprises cooling tissue adjacent to the intermetatarsal neuroma after administering capsaicin. The cooling may involve placing a cooled article (e.g., an icepack) on the surface of the patient's foot having the intermetatarsal neuroma. In certain embodiments, the cooled article may be a device configured for placement on the surface of the patient's foot, where the device contains a cooled fluid, which may be a circulating cooled fluid (e.g., where the circulating cooled fluid has a temperature in the range of about 5° C. to about 10° C., about 10° C. to about 20° C., about 13° C. to about 17° C., or more preferably about 15° C.). The device configured for placement on the surface of the patient's foot may be configured to encompass the patient's foot. The device may be placed on the patient's foot for a duration necessary to achieve the desired amount of tissue cooling. In certain embodiments, the device may be placed on the patient's foot for a duration of about 15 to 30 minutes, about 30 minutes to 60 minutes, about 60 minutes to 90 minutes, or longer.

### Control of Procedure Pain Using a Local Anesthetic Agent

The methods may be further characterized according to administration of a local anesthetic agent to reduce pain experienced by the patient due to injection of the capsaicin. In certain embodiments, the method further comprises administering a local anesthetic agent to the patient immediately prior to injecting the capsaicin in order to ameliorate any pain experienced by the patient due to administering the capsaicin.

The local anesthetic agent may be, for example, a caine analagesic. Exemplary caine analgesics include, for example, lidocaine, dibucaine, bupivacaine, ropivacaine, etidocaine, tetracaine, procaine, chlorocaine, prilocaine, mepivacaine, xylocaine, 2-chloroprocaine, and pharmaceutically acceptable salts thereof. In certain embodiments, the local anesthetic agent is lidocaine or a pharmaceutically acceptable salt thereof.

The dose of local anesthetic will depend on the anesthetic being administered as well as the site where the local anesthetic is administered. For example, in embodiments where the local anesthetic is administered via a regional block (e.g., an ankle block), the dose of anesthetic may range from about 1 mL up to about 30 mL of a 1% solution of anesthetic agent (e.g., lidocaine). In other embodiments, a dose of up to 5 mg/kg of a solution containing 0.25% to 5% of anesthetic agent (e.g., lidocaine) may be administered as a nerve block, such as by administration to the site of pain or an area proximal to the site of pain. In yet other embodiments, the dose of local anesthetic may range from about 0.5 mL to about 60 mL of a 0.25% to 5% solution of anesthetic agent.

The methods may be further characterized according to the location in which the local anesthetic agent is administered. In certain embodiments, the local anesthetic agent is administered to tissue adjacent to the intermetatarsal neuroma. In certain embodiments, the local anesthetic agent is administered to the ankle attached to the patient's foot having the intermetatarsal neuroma.

Alternatively, a general anesthetic (or other agent that causes sedation) may be used to attenuate any initial hyperalgesic effect caused by the administration of capsaicin.

As noted above, multiple features described herein may be combined in a therapeutic method. One example of such a combination is use of (i) a step taken to reduce the temperature of tissue adjacent to the intermetatarsal neuroma before and/or after administration of the capsaicin, together with (ii) administering a local anesthetic agent to reduce pain experienced by the patient due to injection of the capsaicin. A more specific illustration of such a combination is a method wherein (i) tissue adjacent to an intermetatarsal neuroma is cooled (e.g., applying a cold article (such as an article having a temperature of about 5° C. to about 10° C., about 10° C. to about 20° C., about 13° C. to about 17° C., or more preferably about 15° C.) to the surface of the patient's foot have the neuroma for approximately 15 minutes), then (ii) administering a local anesthetic agent to tissue around the intermetatarsal neuroma (e.g., injecting an aqueous solution of lidocaine (which may involve injecting up to, for example, 4 mL of a 1% lidocaine solution)) approximately 30 minutes prior to administering capsaicin to the patient's foot having the intermetatarsal neuroma, and then (iii) applying a cold article (e.g., an article have a temperature of about 5° C. to about 10° C., about 10° C. to about 20° C., about 13° C. to about 17° C., or more preferably about 15° C.) to the surface of the patient's foot having received the capsaicin for a duration of, for example, about 30 minutes to about 60 minutes.

Timing for Administering any Second Dose or Additional Dose of Capsaicin without the Need for Local Anesthetic Agent to Control Procedure Pain

Methods are contemplated in which any second dose or additional dose of capsaicin may be administered to the patient without administering a local anesthetic agent to the patient immediately prior to injecting the capsaicin, and any pain experienced by the patient due to the administration of a second dose or additional dose of capsaicin is no greater than a score of mild on the Injection Pain Scale. It is contemplated that the second dose or additional dose of capsaicin is administered soon enough after a first or prior dose of capsaicin, then pain ablation due to the first dose or prior dose of capsaicin will be sufficient to ameliorate some or all of the pain typically experienced by the patient due to administration of capsaicin. As a more specific illustration, where the duration of the analgesia provided by a dose of capsaicin is greater than 6 months, administration of a subsequent dose of capsaicin every six months permits continuous relief from pain while also minimizing or eliminating any need for a local anesthetic or local cooling of tissue adjacent to the intermetatarsal neuroma in order to alleviate temporary pain associated with administration of capsaicin.

Methods are also contemplated in which any second dose or additional dose of capsaicin may be administered to the patient without administering a local anesthetic agent to the patient immediately prior to injecting the capsaicin, though a step is taken to reduce the temperature of tissue adjacent to the intermetatarsal neuroma before and/or after administration of the capsaicin (e.g., applying a cold article (e.g., an article having a temperature about 5° C. to about 10° C., about 10° C. to about 20° C., about 13° C. to about 17° C., or more preferably about 15° C.) to the surface of the patient's foot having the intermetarsal neuroma to receive the capsaicin), and any pain experienced by the patient due to administration of a second dose or additional dose of capsaicin is no greater than a score of mild on the Injection Pain Scale. Still further, methods are also contemplated in which any second dose or additional dose of capsaicin may be administered to the patient without administering a local anesthetic agent to the patient immediately prior to injecting the capsaicin, no step taken is to reduce the temperature of tissue adjacent to the intermetatarsal neuroma before and/or after administration of the capsaicin (e.g., applying a cold article (e.g., an article having a temperature of about 5° C. to about 10° C., about 10° C. to about 20° C., about 13° C. to about 17° C., or more preferably about 15° C.) to the surface of the patient's foot having the intermetarsal neuroma to receive the capsaicin), and any pain experienced by the patient due to the administration of a second dose or additional dose of capsaicin is no greater than a score of mild on the Injection Pain Scale.

The “Injection Pain Scale” is a measure of pain experienced by a patient upon administration of capsaicin by injection, where the extent of pain experienced by the patient is rated by the patient as one of the following: (i) none, (ii) mild pain, (iii) moderate pain, or (iv) intense pain.

### Location of Intermetatarsal Neuroma

The methods may be further characterized according to the location of the intermetatarsal neuroma. In certain embodiments, the patient has an intermetatarsal neuroma in the third intermetatarsal space. In certain embodiments, the patient has an intermetatarsal neuroma in the second intermetatarsal space.

### Characterization of the Intermetatarsal Neuroma

The methods may be further characterized according to features of the intermetatarsal neuroma, such as numbness in a toe of the foot having the intermetatarsal neuroma, paresthesia in a toe of the foot having the intermetatarsal neuroma, magnitude of pain experienced by the patient due to the intermetatarsal neuroma, and/or size of the intermetatarsal neuroma.

Accordingly, in certain embodiments, the method is further characterized by the feature that the patient experiences numbness in a toe or experiences paresthesia in a toe, each due to the intermetatarsal neuroma.

In certain embodiments, the method is characterized according to the magnitude of pain experienced by the patient due to the intermetatarsal neuroma. In certain embodiments, the patient experiences pain due to the intermetatarsal neuroma of at least a level 4 at some point during the twenty-four hour period prior to administering the first dose of capsaicin. In certain embodiments, the patient experiences pain due to the intermetatarsal neuroma of at least a level 5 at some point during the twenty-four hour period prior to administering the first dose of capsaicin. In certain embodiments, the patient experiences pain due to the intermetatarsal neuroma of at least a level 4 at some point during the twenty-four hour period prior to administering the capsaicin. In certain embodiments, the patient experiences pain due to the intermetatarsal neuroma of at least a level 5 at some point during the twenty-four hour period prior to administering the capsaicin.

In certain embodiments, the method is characterized according to the size of the intermetatarsal neuroma. In certain embodiments, the enlarged nerve of the intermetatarsal neuroma has a diameter of at least 3 mm. In certain embodiments, the enlarged nerve of the intermetatarsal neuroma has a diameter in the range of about 4 mm to about 9 mm. In certain embodiments, the enlarged nerve of the intermetatarsal neuroma has a diameter in the range of about 5 mm to about 8 mm. In certain embodiments, wherein the enlarged nerve of the intermetatarsal neuroma has a diameter in the range of about 5 mm to about 6 mm, about 6 mm to about 7 mm, about 7 mm to about 8 mm, about 8 mm to about 9 mm, or greater than 9 mm.

### Characterization of Pain Reduction Effect of Capsaicin Treatment

The methods may be further characterized according to reduction in pain provided by the capsaicin treatment. For example, in certain embodiments, the method is characterized by achieving a reduction in average walking foot pain due to the intermetatarsal neuroma for a certain duration of time. In certain embodiments, the method is characterized by achieving a reduction in average walking foot pain due to the intermetatarsal neuroma by at least 1 on the Numeric Pain Rating Scale (NPRS) for a duration of at least 3 months. In certain embodiments, the method is characterized by achieving a reduction in average walking foot pain due to the intermetatarsal neuroma by at least 1 on the Numeric Pain Rating Scale (NPRS) for a duration of at least 4 months. In certain embodiments, the method is characterized by achieving a reduction in average walking foot pain due to the intermetatarsal neuroma by at least 1 on the Numeric Pain Rating Scale (NPRS) for a duration of at least 5 months. In certain embodiments, the method is characterized by achieving a reduction in average walking foot pain due to the intermetatarsal neuroma by at least 1 on the Numeric Pain Rating Scale (NPRS) for a duration of at least 6 months. In certain embodiments, the method is characterized by achieving a reduction in average walking foot pain due to the intermetatarsal neuroma by at least 1 on the Numeric Pain Rating Scale (NPRS) for a duration of at least 7 months. In certain embodiments, the method is characterized by achieving a reduction in average walking foot pain due to the intermetatarsal neuroma by at least 1 on the Numeric Pain Rating Scale (NPRS) for a duration of at least 8 months. In certain embodiments, the method is characterized by achieving a reduction in average walking foot pain due to the intermetatarsal neuroma by at least 1 on the Numeric Pain Rating Scale (NPRS) for a duration of at least 9 months. In certain embodiments, the method is characterized by achieving a reduction in average walking foot pain due to the intermetatarsal neuroma by at least 1 on the Numeric Pain Rating Scale (NPRS) for a duration of at least 10 months. In certain embodiments, the method is characterized by achieving a reduction in average walking foot pain due to the intermetatarsal neuroma by at least 1 on the Numeric Pain Rating Scale (NPRS) for a duration of at least 11 months. In certain embodiments, the method is characterized by achieving a reduction in average walking foot pain due to the intermetatarsal neuroma by at least 1 on the Numeric Pain Rating Scale (NPRS) for a duration of at least 12 months. In certain embodiments, the method is characterized by achieving a reduction in average walking foot pain due to the intermetatarsal neuroma by at least 1 on the Numeric Pain Rating Scale (NPRS) for a duration of at least 12 months, where the patient features conditions where nerve growth is delayed in the area of the intermetatarsal neuroma, such as in diabetes mellitus.

In certain embodiments, the method is characterized by achieving a reduction in average walking foot pain due to the intermetatarsal neuroma by at least 2 on the Numeric Pain Rating Scale (NPRS) for a certain duration of time. In certain embodiments, the method is characterized by achieving a reduction in average walking foot pain due to the intermetatarsal neuroma by at least 2 on the Numeric Pain Rating Scale (NPRS) for a duration of at least 3 months. In certain embodiments, the method is characterized by achieving a reduction in average walking foot pain due to the intermetatarsal neuroma by at least 2 on the Numeric Pain Rating Scale (NPRS) for a duration of at least 4 months. In certain embodiments, wherein the method is characterized by achieving a reduction in average walking foot pain due to the intermetatarsal neuroma by at least 2 on the Numeric Pain Rating Scale (NPRS) for a duration of at least 5 months. In certain embodiments, wherein the method is characterized by achieving a reduction in average walking foot pain due to the intermetatarsal neuroma by at least 2 on the Numeric Pain Rating Scale (NPRS) for a duration of at least 6 months. In certain embodiments, wherein the method is characterized by achieving a reduction in average walking foot pain due to the intermetatarsal neuroma by at least 2 on the Numeric Pain Rating Scale (NPRS) for a duration of at least 7 months. In certain embodiments, wherein the method is characterized by achieving a reduction in average walking foot pain due to the intermetatarsal neuroma by at least 2 on the Numeric Pain Rating Scale (NPRS) for a duration of at least 8 months. In certain embodiments, wherein the method is characterized by achieving a reduction in average walking foot pain due to the intermetatarsal neuroma by at least 2 on the Numeric Pain Rating Scale (NPRS) for a duration of at least 9 months. In certain embodiments, wherein the method is characterized by achieving a reduction in average walking foot pain due to the intermetatarsal neuroma by at least 2 on the Numeric Pain Rating Scale (NPRS) for a duration of at least 10 months. In certain embodiments, wherein the method is characterized by achieving a reduction in average walking foot pain due to the intermetatarsal neuroma by at least 2 on the Numeric Pain Rating Scale (NPRS) for a duration of at least 11 months. In certain embodiments, wherein the method is characterized by achieving a reduction in average walking foot pain due to the intermetatarsal neuroma by at least 2 on the Numeric Pain Rating Scale (NPRS) for a duration of at least 12 months. In certain embodiments, wherein the method is characterized by achieving a reduction in average walking foot pain due to the intermetatarsal neuroma by at least 2 on the Numeric Pain Rating Scale (NPRS) for a duration of at least 12 months, where the patient features conditions where nerve growth is delayed in the area of the intermetatarsal neuroma, such as in diabetes mellitus.

The methods may be further characterized according to the maximal amount of pain experienced by the patient due to the intermetatarsal neuroma following administration of capsaicin. For example, in certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for certain durations of time, such as at least 1, 2, 3, 4, 5, 6, 7, 8, 9, 10, 11, or 12 months. Accordingly, in certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 3 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 4 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 5 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 6 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 7 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 8 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 9 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 10 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 11 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 12 months. In certain embodiments, the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 12 months, where the patient features conditions where nerve growth is delayed in the area of the intermetatarsal neuroma, such as in diabetes mellitus. In yet other embodiments, the method is characterized by reducing the patient's average walking foot pain due to the intermetatarsal neuroma so that the patient's average walking foot pain due to the intermetatarsal neuroma is no greater than 2 on the Numeric Pain Rating Scale (NPRS) for certain durations of time, such as at least 1, 2, 3, 4, 5, 6, 7, 8, 9, 10, 11, or 12 months. In yet other embodiments, the method is characterized by reducing the patient's average walking foot pain due to the intermetatarsal neuroma so that the patient's average walking foot pain due to the intermetatarsal neuroma is no greater than 3 on the Numeric Pain Rating Scale (NPRS) for certain durations of time, such as at least 1, 2, 3, 4, 5, 6, 7, 8, 9, 10, 11, or 12 months. In yet other embodiments, the method is characterized by reducing the patient's average walking foot pain due to the intermetatarsal neuroma so that the patient's average walking foot pain due to the intermetatarsal neuroma is no greater than 4 on the Numeric Pain Rating Scale (NPRS) for certain durations of time, such as at least 1, 2, 3, 4, 5, 6, 7, 8, 9, 10, 11, or 12 months. In yet other embodiments, the method is characterized by reducing the patient's average walking foot pain due to the intermetatarsal neuroma so that the patient's average walking foot pain due to the intermetatarsal neuroma is no greater than 5 on the Numeric Pain Rating Scale (NPRS) for certain durations of time, such as at least 1, 2, 3, 4, 5, 6, 7, 8, 9, 10, 11, or 12 months.

The methods may be further characterized according to the reduction in pain experienced by the patient due to the intermetatarsal neuroma following administration of a first dose of capsaicin. Accordingly, in certain embodiments, the method is characterized by the feature that upon administration of the first dose of capsaicin, the patient experiences a reduction in average walking foot pain due to the intermetatarsal neuroma of at least 1 on the Numeric Pain Rating Scale (NPRS) within 2 weeks after administration of the first dose of capsaicin and lasting for a duration of at least 2 months. In certain embodiments, wherein upon administration of the first dose of capsaicin, the patient experiences a reduction in average walking foot pain due to the intermetatarsal neuroma of at least 2 on the Numeric Pain Rating Scale (NPRS) within 2 weeks after administration of the first dose of capsaicin and lasting for a duration of at least 2 months. In certain embodiments, wherein upon administration of the first dose of capsaicin, the patient experiences a reduction in average walking foot pain due to the intermetatarsal neuroma of at least 1 on the Numeric Pain Rating Scale (NPRS) within 2 weeks after administration of the first dose of capsaicin and lasting for a duration of at least 3 months. In certain embodiments, wherein upon administration of the first dose of capsaicin, the patient experiences a reduction in average walking foot pain due to the intermetatarsal neuroma of at least 2 on the Numeric Pain Rating Scale (NPRS) within 2 weeks after administration of the first dose of capsaicin and lasting for a duration of at least 3 months.

The methods may be further characterized according to ability to reduce the patient's worst neuroma foot pain due to the intermetatarsal neuroma so that the patient's worst neuroma foot pain due to the intermetatarsal neuroma is no greater than 1 on the Numeric Pain Rating Scale for certain duration of time, such as at least 1, 2, 3, 4, 5, 6, 7, 8, 9, 10, 11, or 12 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 3 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 4 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 5 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 6 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 7 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 8 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 9 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 10 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 11 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 12 months. In certain embodiments, the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 12 months, where the patient features conditions where nerve growth is delayed in the area of the intermetatarsal neuroma, such as in diabetes mellitus.

The methods may be further characterized according to ability to reduce the patient's worst neuroma foot pain due to the intermetatarsal neuroma so that the patient's worst neuroma foot pain due to the intermetatarsal neuroma is no greater than a certain threshold (e.g., 1 or 2) on the Numeric Pain Rating Scale for certain duration of time after administering the first dose of capsaicin, such as at least 1, 2, 3, 4, 5, 6, 7, 8, 9, 10, 11, or 12 months. In certain embodiments, upon administration of the first dose of capsaicin, the patient experiences a reduction in worst neuroma foot pain due to the intermetatarsal neuroma of at least 1 on the Numeric Pain Rating Scale (NPRS) within 2 weeks after administration of the first dose of capsaicin and lasting for a duration of at least 2 months. In certain embodiments, upon administration of a said dose of capsaicin, the patient experiences a reduction in worst neuroma foot pain due to the intermetatarsal neuroma of at least 2 on the Numeric Pain Rating Scale (NPRS) within 2 weeks after administration of the first dose of capsaicin and lasting for a duration of at least 2 months. In certain embodiments, upon administration of a said dose of capsaicin, the patient experiences a reduction in worst neuroma foot pain due to the intermetatarsal neuroma of at least 1 on the Numeric Pain Rating Scale (NPRS) within 2 weeks after administration of the first dose of capsaicin and lasting for a duration of at least 3 months. In certain embodiments, upon administration of a said dose of capsaicin, the patient experiences a reduction in worst neuroma foot pain due to the intermetatarsal neuroma of at least 2 on the Numeric Pain Rating Scale (NPRS) within 2 weeks after administration of the first dose of capsaicin and lasting for a duration of at least 3 months.

The methods may be further characterized according to ability to achieve an improvement in the patient's Revised Foot Function Index (FFI-R) score. Accordingly, in certain embodiments, upon administration of a first dose of capsaicin, the patient experiences an improvement in their Revised Foot Function Index (FFI-R) score of at least 1 within 2 weeks after administration of the dose of capsaicin and lasting for a duration of at least 2 months. In certain embodiments, upon administration of a said dose of capsaicin, the patient experiences an improvement in their Revised Foot Function Index (FFI-R) score of at least 2 within 2 weeks after administration of the dose of capsaicin and lasting for a duration of at least 2 months. In certain embodiments, upon administration of a said dose of capsaicin the patient experiences an improvement in their Revised Foot Function Index (FFI-R) score of at least 1 within 2 weeks after administration of the dose of capsaicin and lasting for a duration of at least 3 months. In certain embodiments, upon administration of a said dose of capsaicin, the patient experiences an improvement in their Revised Foot Function Index (FFI-R) score of at least 2 within 2 weeks after administration of the dose of capsaicin and lasting for a duration of at least 2 months. In certain embodiments, the method is characterized by the patient experiencing an improvement in their Revised Foot Function Index (FFI-R) score of at least 1 (or at least 2 or 3) for a duration of at least 2, 3, 4, 5, 6, 7, 8, 9, 10, 11, or 12 months.

The methods may be further characterized according to ability to achieve an improvement in the patient's Personalized Activity Rating Scale (PARS) score. In certain embodiments, upon administration of a said dose of capsaicin, the patient experiences an improvement in their Personalized Activity Rating Scale (PARS) score of at least 1 within 2 weeks after administration of the dose of capsaicin and lasting for a duration of at least 1 month. In certain embodiments, wherein upon administration of a said dose of capsaicin, the patient experiences an improvement in their Personalized Activity Rating Scale (PARS) score of at least 2 within 2 weeks after administration of the dose of capsaicin and lasting for a duration of at least 1 month. In certain embodiments, wherein upon administration of a said dose of capsaicin the patient experiences an improvement in their Personalized Activity Rating Scale (PARS) score of at least 1 within 2 weeks after administration of the dose of capsaicin and lasting for a duration of at least 2 months. In certain embodiments, wherein upon administration of a said dose of capsaicin, the patient experiences an improvement in their Personalized Activity Rating Scale (PARS) score of at least 2 within 2 weeks after administration of the dose of capsaicin and lasting for a duration of at least 2 months. In certain embodiments, the method is characterized by the patient experiencing an improvement in their Personalized Activity Rating Scale (PARS) score of at least 1 (or at least 2 or 3) for a duration of at least 2, 3, 4, 5, 6, 7, 8, 9, 10, 11, or 12 months.

The methods may be further characterized according to improvements in the patient's quality of life following administration of capsaicin to ameliorate pain due to the intermetatarsal neuroma. For example, in certain embodiments, the method is characterized by an improvement in the patient's Quality of Life score, such as an improvement on a EuroQol-5 Dimensions (EQ-5D-5L) scale.

The methods may be further characterized according to the magnitude of the reduction in pain produced by administration of the second dose and any subsequent dose of capsaicin. For example, in certain embodiments, administration of the second dose of capsaicin achieves a reduction in pain greater than the reduction in pain achieved by administration of the first dose of capsaicin, wherein the amount of capsaicin in the second dose is no greater than the amount of capsaicin in the first dose, and the reduction in pain is measured by comparing (i) the amount of pain due to the intermetatarsal neuroma experienced by the patient just prior to administering the dose of capsaicin to (ii) the amount of pain due to the intermetatarsal neuroma experienced by the patient at a time that is four weeks after administering said dose of capsaicin. In other embodiments, administration of the second dose of capsaicin achieves a reduction in pain that is at least fifty percent of the reduction in pain achieved by administration of the first dose of capsaicin, wherein the amount of capsaicin in the second dose is no greater than the amount of capsaicin in the first dose, and the reduction in pain is measured by comparing (i) the amount of pain due to the intermetatarsal neuroma experienced by the patient just prior to administering the dose of capsaicin to (ii) the amount of pain due to the intermetatarsal neuroma experienced by the patient at a time that is four weeks after administering said dose of capsaicin. In other embodiments, administration of a third dose of capsaicin achieves a reduction in pain greater than the reduction in pain achieved by administration of the second dose of capsaicin, wherein the amount of capsaicin in the third dose is no greater than the amount of capsaicin in the second dose, and the reduction in pain is measured by comparing (i) the amount of pain due to the intermetatarsal neuroma experienced by the patient just prior to administering the dose of capsaicin to (ii) the amount of pain due to the intermetatarsal neuroma experienced by the patient at a time that is four weeks after administering said dose of capsaicin. In other embodiments, administration of a third dose of capsaicin achieves a reduction in pain that is at least fifty percent of the reduction in pain achieved by administration of the second dose of capsaicin, wherein the amount of capsaicin in the third dose is no greater than the amount of capsaicin in the second dose, and the reduction in pain is measured by comparing (i) the amount of pain due to the intermetatarsal neuroma experienced by the patient just prior to administering the dose of capsaicin to (ii) the amount of pain due to the intermetatarsal neuroma experienced by the patient at a time that is four weeks after administering said dose of capsaicin.

### Patient Populations for Treatment

The methods may be further characterized according to features of the patients to be treated. For example, in certain embodiments, during the 24 hour period prior to administration of the first dose of capsaicin, the patient suffers from one or more of the following: (a) an average walking foot pain due to the intermetatarsal neuroma of at least 4 on the Numeric Pain Rating Scale (NPRS); (b) a worst neuroma foot pain due to the intermetatarsal neuroma of at least 4 on the Numeric Pain Rating Scale (NPRS); or (c) a Revised Foot Function Index (FFI-R) score indicating the patient experiences at least two of the following: (i) moderate pain due to the intermetatarsal neuroma, (ii) moderate stiffness due to the intermetatarsal neuroma, and (iii) moderate difficulty in a physical activity due to the intermetatarsal neuroma. In certain other embodiments, during the 24 hour period prior to administration of the first dose of capsaicin, the patient suffers from one or more of the following: (a) an average walking foot pain due to the intermetatarsal neuroma of at least 6 on the Numeric Pain Rating Scale (NPRS); (b) a worst neuroma foot pain due to the intermetatarsal neuroma of at least 6 on the Numeric Pain Rating Scale (NPRS); or (c) a Revised Foot Function Index (FFI-R) score indicating the patient experiences at least two of the following: (i) severe pain due to the intermetatarsal neuroma, (ii) severe stiffness due to the intermetatarsal neuroma, and (iii) severe difficulty in a physical activity due to the intermetatarsal neuroma. In certain other embodiments, during the 24 hour period prior to administration of the first dose of capsaicin, the patient suffers from one or more of the following: (a) an average walking foot pain due to the intermetatarsal neuroma of at least 8 on the Numeric Pain Rating Scale (NPRS); (b) a worst neuroma foot pain due to the intermetatarsal neuroma of at least 8 on the Numeric Pain Rating Scale (NPRS); or (c) a Revised Foot Function Index (FFI-R) score indicating the patient experiences at all of the following: (i) severe pain due to the intermetatarsal neuroma, (ii) severe stiffness due to the intermetatarsal neuroma, and (iii) severe difficulty in a physical activity due to the intermetatarsal neuroma.

In certain embodiments, the patient is characterized according to one or more of: average walking foot pain due to the intermetatarsal neuroma, worst neuroma foot pain due to the intermetatarsal neuroma, Revised Foot Function Index (FFI-R) score, and Personalized Activity Rating Scale (PARS). Accordingly, in certain embodiments, during the 24 hour period prior to administration of the first dose of capsaicin, the patient suffers from one or more of the following: (a) an average walking foot pain due to the intermetatarsal neuroma of at least 4 on the Numeric Pain Rating Scale (NPRS); (b) a worst neuroma foot pain due to the intermetatarsal neuroma of at least 4 on the Numeric Pain Rating Scale (NPRS); (c) a Revised Foot Function Index (FFI-R) score indicating the patient experiences at least two of the following: (i) moderate pain due to the intermetatarsal neuroma, (ii) moderate stiffness due to the intermetatarsal neuroma, and (iii) moderate difficulty in a physical activity due to the intermetatarsal neuroma; or (d) a Personalized Activity Rating Scale (PARS) score of at least 4 for at least one physical activity. In certain embodiments, during the 24 hour period prior to administration of the first dose of capsaicin, the patient suffers from one or more of the following: (a) an average walking foot pain due to the intermetatarsal neuroma of at least 6 on the Numeric Pain Rating Scale (NPRS); (b) a worst neuroma foot pain due to the intermetatarsal neuroma of at least 6 on the Numeric Pain Rating Scale (NPRS); (c) a Revised Foot Function Index (FFI-R) score indicating the patient experiences at least two of the following: (i) severe pain due to the intermetatarsal neuroma, (ii) severe stiffness due to the intermetatarsal neuroma, and (iii) severe difficulty in a physical activity due to the intermetatarsal neuroma; or (d) a Personalized Activity Rating Scale (PARS) score of at least 6 for at least one physical activity. In certain embodiments, during the 24 hour period prior to administration of the first dose of capsaicin, the patient suffers from one or more of the following: (a) an average walking foot pain due to the intermetatarsal neuroma of at least 8 on the Numeric Pain Rating Scale (NPRS); (b) a worst neuroma foot pain due to the intermetatarsal neuroma of at least 8 on the Numeric Pain Rating Scale (NPRS); (c) a Revised Foot Function Index (FFI-R) score indicating the patient experiences at all of the following: (i) severe pain due to the intermetatarsal neuroma, (ii) severe stiffness due to the intermetatarsal neuroma, and (iii) severe difficulty in a physical activity due to the intermetatarsal neuroma; or (d) a Personalized Activity Rating Scale (PARS) score of at least 8 for at least one physical activity.

The methods may be further characterized according to whether the patient has a low Quality of Life score, such as a low score on a EuroQol-5 Dimensions (EQ-5D-5L) scale, due to pain or other conditions due to the intermetatarsal neuroma.

The methods may be further characterized according to whether the patient was previously able to achieve temporarily relief from the pain due to intermetatarsal neuroma using other therapies, such as an injectable steroid, an oral analgesic, or sclerosing agent. Accordingly, in certain embodiments, the method is further characterized by the feature that the patient did not achieve relief from pain due the intermetatarsal neuroma for a duration greater than 2 months following treatment using an injectable steroid, an oral analgesic, or administration of a sclerosing agent to alleviate pain due to the intermetatarsal neuroma.

The methods may be further characterized according to the age of the patient. In certain embodiments, the patient has an age in the range of about 20 to about 30 years old, about 30 to about 40 years old, about 40 to about 50 years old, about 50 to about 60 years old, or about 60 to about 70 years old, or an age greater than 70 years old.

The methods may be further characterized according to the gender of the patient, such as a male or female patient. In certain embodiments, the patient is an adult human male, or an adult human female. In certain embodiments, the patient is a transgender human.

In certain embodiments, the patient is a pediatric human.

### Exemplary More Specific Methods

As explained above, features described herein may be combined to provide a more specific method. One exemplary more specific method is a method of ameliorating pain for a duration of at least 12 months due to an intermetatarsal neuroma in a patient, where the method comprises administering by injection into the patient's intermetatarsal space having an intermetatarsal neuroma at least a first dose of capsaicin and a second dose of capsaicin to ameliorate pain due to the intermetatarsal neuroma for a duration of at least 12 months, wherein (a) the first dose of capsaicin is in an amount ranging from about 100 μg to about 1,000 μg of capsaicin; (b) the second dose of capsaicin is in an amount ranging from about 100 μg to about 1,000 μg of capsaicin; (c) the second dose of capsaicin is administered about 6 months after administration of the first dose of capsaicin; and (d) any additional dose of capsaicin is administered by injection into the patient's intermetatarsal space having an intermetatarsal neuroma, any such additional dose is in an amount ranging from about 100 μg to about 1,000 μg of capsaicin and any said additional dose is administered about 6 months after administration of the prior dose of capsaicin administered by injection into the patient's intermetatarsal space having an intermetatarsal neuroma. In certain embodiments, at least 2, 3, 4, 5, or 6 additional doses of capsaicin are administered. In certain embodiments, additional doses of capsaicin are administered to the patient on a repeating basis in order to achieve continued amelioration of pain due to the intermetatarsal neuroma. In a preferred embodiment, the first dose of capsaicin is about 200 μg of capsaicin, the second dose of capsaicin is about 200 μg of capsaicin, and each additional dose of capsaicin is about 200 μg of capsaicin. The capsaicin is preferably administered as an injectable solution containing water and a poly(ethylene glycol), wherein the injectable solution has a volume of about 2 mL.

### III. Injectable Formulations

Various injectable formulations are described in the literature and known to those of skill in the art. The injectable formulation may typically contain water and one or more additional components to render the formulation optimally suited for injection into a subject.

When administering capsaicin according to methods described herein, the capsaicin is desirably administered in the form of a pharmaceutical composition formulated for injection. In certain embodiments, the pharmaceutical composition formulated for injection is an aqueous pharmaceutical composition.

The capsaicin may be dissolved in oils, polyethylene glycol (PEG), propylene glycol (PG), and/or other solvents commonly used to prepare injectable or implantable solutions. Suitable pharmaceutically acceptable vehicles include aqueous vehicles, nonaqueous vehicles, antimicrobial agents, isotonic agents, buffers, antioxidants, suspending and dispersing agents, emulsifying agents, sequestering or chelating agents, and combinations or mixtures thereof. It is appreciated that when one or more solvents are used in the formulations of the invention, they may be combined, e.g., with a pharmaceutically acceptable buffer and may be present in the final formulation, e.g., in an amount ranging from about 10% to about 100%, more preferably from about 20% to about 100%.

Exemplary aqueous vehicles include Sodium Chloride Injection, Bacteriostatic Sodium Chloride Injection, Ringers Injection, Isotonic Dextrose Injection, Sterile Water Injection, Bacteriostatic Sterile Water Injection, Dextrose Lactated Ringers Injection and any combinations or mixtures thereof.

Exemplary nonaqueous parenteral vehicles include fixed oils of vegetable origin, cottonseed oil, corn oil, sesame oil, peanut oil, and combinations or mixtures thereof.

Exemplary antimicrobial agents in bacteriostatic or fungistatic concentrations include phenols, cresols, mercurials, benzyl alcohol, chlorobutanol, ethyl and propyl p-hydroxybenzoic acid esters, thimerosal, benzalkonium chloride, benzethonium chloride, and mixtures thereof.

Exemplary isotonic agents include sodium chloride, dextrose, and combinations or mixtures thereof.

Exemplary antioxidants include ascorbic acid, sodium bisulfate, and combinations or mixtures thereof.

Exemplary suspending and dispersing agents include sodium carboxymethylcelluose, hydroxypropyl methylcellulose, polyvinylpyrrolidone, any combinations or mixtures thereof.

Exemplary emulsifying agents include anionic emulsifying agents (e.g., sodium lauryl sulfate, sodium stearate, calcium oleate, and combinations or mixtures thereof), cationic emulsifying agents (e.g., cetrimide), and non-ionic emulsifying agents (e.g., Polysorbate 80 (Tween 80)).

Exemplary sequestering or chelating agents of metal ions include ethylenediaminetetraacetic acid (EDTA), citric acid, sorbitol, tartaric acid, phosphoric acid, and the like.

Suitable surfactants include, but are not limited to, sodium stearyl fumarate, diethanolamine cetyl sulfate, polyethylene glycol, isostearate, polyethoxylated castor oil, benzalkonium chloride, nonoxyl 10, octoxynol 9, polyoxyethylene sorbitan fatty acids (polysorbate 20, 40, 60 and 80), sodium lauryl sulfate, sorbitan esters (sorbitan monolaurate, sorbitan monooleate, sorbitan monopalmitate, sorbitan monostearate, sorbitan sesquioleate, sorbitan trioleate, sorbitan tristearate, sorbitan laurate, sorbitan oleate, sorbitan palmitate, sorbitan stearate, sorbitan dioleate, sorbitan sesqui-isostearate, sorbitan sesquistearate, sorbitan tri-isostearate), lecithin pharmaceutical acceptable salts thereof and combinations thereof. When one or more surfactants are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%. In certain other embodiments, a surfactant can preferably be combined with one or more of the pharmaceutically acceptable vehicles previously described herein so that the surfactant or buffering agent prevents the initial stinging or burning discomfort associated with capsaicinoid administration, as a wetting agent, emulsifier, solubilizer and/or antimicrobial.

Buffering agents may also be used to provide drug stability; to control the therapeutic activity of the drug substance (Ansel, Howard C., “Introduction to Pharmaceutical Dosage Forms,” 4^{th} Ed., 1985); and/or to prevent the initial stinging or burning discomfort associated with capsaicin administration. Suitable buffers include, but are not limited to, sodium bicarbonate, sodium citrate, citric acid, sodium phosphate, pharmaceutically acceptable salts thereof, and combinations thereof. When one or more buffers are utilized in the formulations of the invention, they may be combined, e.g., with a pharmaceutically acceptable vehicle and may be present in the final formulation, e.g., in an amount ranging from about 0.1% to about 20%, more preferably from about 0.5% to about 10%. In certain embodiments, the buffer is an acetate salt, phosphate salt, citrate salt; corresponding acids of the foregoing; and combinations or mixtures thereof.

In certain embodiments, the pharmaceutical vehicle utilized to deliver the injectable capsaicin may comprise about 20% PEG 300, about 10 mM histidine and about 5% sucrose in water for injection. In certain other embodiments, the pharmaceutical vehicle utilized to deliver the injectable capsaicin may comprise about 30-50% PEG 300. This may be used as such or further diluted in water for injection to achieve a larger volume.

The injectable formulation may be further characterized according to the concentration of capsaicin in the formulation. In certain embodiments, the injectable formulation contains the capsaicin at a concentration ranging from about 0.01 mg/mL to about 4 mg/mL, about 0.05 mg/mL to about 3 mg/mL, about 0.1 mg/mL to about 2 mg/mL, about 0.15 mg/mL to about 2 mg/mL, about 0.2 mg/mL to about 0.8 mg/mL, about 0.25 mg/mL to about 0.6 mg/mL, about 0.25 mg/mL to about 0.5 mg/mL, about 0.3 mg/mL to about 0.5 mg/mL, about 0.3 mg/mL to about 0.4 mg/mL, about 0.35 mg/mL to about 0.45 mg/mL, or about 0.375 mg/mL to about 0.425 mg/mL. In certain preferred embodiments, the injectable formulation contains capsaicin at a concentration ranging from about 0.05 mg/mL to about 0.15 mg/mL, or about 0.3 mg/mL to about 0.4 mg/mL. In certain other preferred embodiments, the injectable formulation contains capsaicin at a concentration of about 0.1 mg/mL.

In certain embodiments, the injectable formulation contains trans-capsaicin at a concentration ranging from about 0.01 mg/mL to about 4 mg/mL, about 0.05 mg/mL to about 3 mg/mL, about 0.1 mg/mL to about 2 mg/mL, about 0.15 mg/mL to about 2 mg/mL, about 0.2 mg/mL to about 0.8 mg/mL, about 0.25 mg/mL to about 0.6 mg/mL, about 0.25 mg/mL to about 0.5 mg/mL, about 0.3 mg/mL to about 0.5 mg/mL, about 0.3 mg/mL to about 0.4 mg/mL, about 0.35 mg/mL to about 0.45 mg/mL, or about 0.375 mg/mL to about 0.425 mg/mL. In certain preferred embodiments, the injectable formulation contains trans-capsaicin at a concentration ranging from about 0.05 mg/mL to about 0.15 mg/mL, or about 0.3 mg/mL to about 0.4 mg/mL. In certain other preferred embodiments, the injectable formulation contains trans-capsaicin at a concentration of about 0.1 mg/mL.

In certain embodiments, the injectable formulation contains the capsaicin at a concentration of about 0.1 mg/mL, 0.15 mg/mL, 0.2 mg/mL, 0.25 mg/mL, 0.3 mg/mL, 0.325 mg/mL, 0.35 mg/mL, 0.37 mg/mL, 0.38 mg/mL, 0.39 mg/mL, 0.4 mg/mL, 0.41 mg/mL, 0.42 mg/mL, 0.43 mg/mL, 0.44 mg/mL, 0.45 mg/mL, 0.475 mg/mL, 0.5 mg/mL, 0.55 mg/mL, 0.575 mg/mL, 0.6 mg/mL, 0.625 mg/mL, 0.65 mg/mL, 0.675 mg/mL, 0.7 mg/mL, 0.75 mg/mL, 0.8 mg/mL, 0.9 mg/mL, 1.0 mg/mL, 1.5 mg/mL, or 2.0 mg/mL. In certain preferred embodiments, the injectable formulation contains the capsaicin at a concentration of about 0.1 mg/mL.

The injectable formulation may be further characterized according to the solvent present to dissolve the capsaicin. In certain embodiments, the solvent in the injectable formulation is a mixture of water and polyethylene glycol (e.g., polyethylene glycol having a number-average molecular weight of about 300 g/mol). The relative amounts of water and polyethylene glycol in the injectable formulation may be characterized. For example, in certain embodiments, the injectable formulation contains a mixture of water and polyethylene glycol (e.g., polyethylene glycol having a number-average molecular weight of about 300 g/mol) as solvent, wherein upon a volume basis there is 3-6 times more water than polyethylene glycol. In certain embodiments, the injectable formulation contains a mixture of water and polyethylene glycol (e.g., polyethylene glycol having a number-average molecular weight of about 300 g/mol) as solvent, wherein upon a volume basis there is 4-5 times more water than polyethylene glycol. In certain embodiments, the polyethylene glycol has a number-average molecular weight in the range of about 250 g/mol to about 350 g/mol.

The injectable formulation may be further characterized according to the volume of injectable formulation administered to tissue proximal to the intermetatarsal neuroma. In certain embodiments, the volume of injectable formulation administered per unit dose is in the range of about 0.5 mL to about 5 mL, about 0.6 mL to about 4 mL, about 0.7 mL to about 3 mL, about 0.8 mL to about 2.5 mL, or about 1 mL to about 2 mL. In certain other embodiments, the volume of injectable formulation administered per unit dose is in the range of about 1.5 mL to about 2.5 mL. In certain other embodiments, the volume of injectable formulation administered per unit dose is about 2 mL.

The foregoing embodiments, may be combined to describe more specific injectable formulations. For example, in certain embodiments, the injectable formulation comprises trans-capsaicin at a concentration of about 0.1 mg/mL, water, and a polyethylene glycol (e.g., polyethylene glycol having a number-average molecular weight of 300 g/mol). In certain embodiments, the injectable formulation comprises trans-capsaicin at a concentration of about 0.1 mg/mL, water, and a polyethylene glycol having a number-average molecular weight of 300 g/mol), wherein upon a volume basis there is 4-5 times more water than polyethylene glycol. In certain embodiments, the injectable formulation consists essentially of trans-capsaicin at a concentration of about 0.1 mg/mL, water, and a polyethylene glycol having a number-average molecular weight of 300 g/mol, wherein upon a volume basis there is 4-5 times more water than polyethylene glycol.

### EXAMPLES

The invention now being generally described, will be more readily understood by reference to the following examples, which are included merely for purposes of illustration of certain aspects and embodiments of the present invention, and is not intended to limit the invention.

### Example 1—Sequential Injection of Capsaicin to Achieve Long Duration Relief from Pain Associated with an Intermetatarsal Neuroma

Patients experiencing pain due to an intermetatarsal neuroma are to be treated by administering up to four doses of trans-capsaicin, at 200 μg of capsaicin per dose, by injecting trans-capsaicin into the area of the neuroma (but not inserting the medical instrument performing the injection into the intermetatarsal neuroma itself). Following the first dose of trans-capsaicin, any subsequent dose of trans-capsaicin is to be administered no sooner than 3 months following the prior dose of trans-capsaicin. Further description of experimental procedures and methods for analysis of pain relief are provided below.

### Patients to be Treated

Patients to be treated are those having previously received trans-capsaicin for relief of pain due to an intermetatarsal neuroma. Patients may receive trans-capsaicin injection in the current study under the following conditions:
1. If the previous injection with trans-capsaicin occurred at least 6 months previously, and the average (walking) neuroma pain has been ≥2 for 2 consecutive interactive web response system (IWRS) or interactive voice response system (IVRS) assessments, or
2. If the previous injection with trans-capsaicin occurred ≥3, but <6 months previously and the patient reports an average (walking) neuroma pain of ≥4 for 2 consecutive IWRS/IVRS assessments.

### Administration of Trans-Capsaicin

trans-Capsaicin is to be injected in the amount of 200 μg per dose by ultrasound-guided needle placement into the area of the neuroma. The dose of trans-capsaicin is injected as a 2 mL solution containing trans-capsaicin at a concentration of 100 μg/mL. Local anesthesia will be performed with up to 4 mL of 1% lidocaine (without epinephrine) injected adjacent to the neuroma 30 minutes prior to injection of trans-capsaicin. Adjunct use of cooling will be applied for 15 minutes before 1% lidocaine injection; after lidocaine injection cooling will be put back on for 30 minutes prior to trans-capsaicin injection. Cooling will be removed for trans-capsaicin injection and then reapplied immediately following the injection for a minimum of 30 minutes and up to 1 hour.

If procedure pain is adequately controlled by the above protocol, subsequent injections will be performed similarly. If the above protocol does not adequately control procedure pain, subsequent trans-capsaicin injections may add an ankle block using an injection of 1% lidocaine such that the posterior tibial nerve at the level of the ankle and the branches of the superficial peroneal nerve on the dorsum of the foot are blocked to achieve a complete sensory blockade in the affected space both dorsal and plantar to the neuroma.

trans-Capsaicin is supplied as a 2 mg/mL solution in PEG-300 (poly ethylene glycol having a number-average molecular weight of approximately 300 g/mol) and must be diluted prior to injection. trans-Capsaicin will be diluted with sterile water and PEG-300 such that the final solution for injection contains 30% PEG-300 at a final concentration of 100 μg/mL trans-capsaicin.

### Study Periods and Visits

Patients are to participate in a Screening/Enrollment visit, Monthly Monitoring visits and phone calls (in alternating months), up to 4 Treatment Cycles which will consist of 4 visits each, and a Week 52/End of Treatment visit. Each Treatment Cycle will be comprised of the following 4 visits: Treatment Visit 1/Treatment Day 1, Treatment Visit 2/Week 1 Phone Call, Treatment Visit 3/Week 2 Clinic visit, and Treatment Visit 4/Week 4 Clinic visit. A Treatment Cycle will begin on the day a subject is scheduled to receive an injection of trans-capsaicin.

Subjects will be eligible to receive additional treatment with trans-capsaicin 200 μg starting at the Enrollment Visit through Week 48 of the study. During this time, if subjects meet the requirements for receiving an injection of trans-capsaicin for their neuroma pain, then they will begin a new Treatment Cycle as described above. Subjects may receive a maximum of 4 treatments with a minimum of 3 months between each dose.

### Screening/Enrollment Visit

The following procedures will be performed at Screening:
1. Written informed consent.
2. Eligibility criteria.
3. Enrollment.
4. Medical history.
5. Complete physical examination (excluding a genitourinary exam) including weight and height.
6. 12-lead electrocardiogram (ECG).
7. Clinical laboratory tests: chemistry, hematology, urinalysis.
8. Urine drug screen.
9. Urine pregnancy test for females of childbearing potential.
10. Vital signs.
11. Training and instruction on assessment of neuroma foot pain (NPRS) during the previous 24 hours and weekly use of the IWRS/IVRS System (NPRS scores and use of rescue medication).
12. Neuroma foot pain at study visit (average walking pain and worst pain over last 24 hours) using NPRS.
13. Foot function assessment.
14. Quality of Life (QoL) assessment.
15. Concomitant medications and therapies. During the study, all medications and non-drug therapies (including rescue medication) be recorded.

### Monthly Monitoring: Telephone Calls and Site Visits

All subjects will record their neuroma foot pain scores and use of rescue medication weekly via IWRS/IVRS system from home throughout the study.

Subjects will be monitored during the course of the study by telephone calls and clinic visits performed on alternating months (i.e., phone call at Month 1, clinic visit at Month 2, phone call at Month 3, etc.). In each monitoring call the subject will be asked assessments.

The first telephone call will take place 4 weeks following the Enrollment/Screening visit and 4 weeks after the Treatment Visit 4/Treatment Week 4 of each Treatment Cycle. The first clinic visit will occur 1 month after the first telephone call.

When subjects have eligible pain as noted above, and receive study treatment, they will complete Treatment Cycle Visits 1 to 4 and then enter post-treatment monitoring. Subjects will receive post-treatment telephone calls every other month and will also return to the clinic during alternating months (every other month).

### Monthly Telephone Calls

During telephone calls, the following assessments will be completed:
1. Adverse events.
2. Concomitant medications and therapies. Details of all medications and non-drug therapies (including rescue medication) will be recorded at this time.
3. Review IWRS/IVRS System compliance with subject, and instruct subject to continue weekly entries (NPRS scores and use of rescue medication). Conduct subject retraining if non-compliant.

### Monthly Site Visits

During in-clinic study visits, the following assessments will be completed:
1. Vital signs.
2. Sensory and motor examination of both feet.
3. Review IWRS/IVRS System entries and compliance with subject, and instruct subject to continue weekly entries (NPRS scores and use of rescue medication). Conduct subject retraining if non-compliant.
4. Neuroma foot pain at study visit (average walking pain and worst pain over last 24 hours) using NPRS.
5. Neuroma foot pain: PGIC from the subject's most recent assessment.
6. Foot function assessment.
7. QoL assessment.
8. Adverse events.
9. Concomitant medications and therapies. Use of all medications and non-drug therapies (including rescue medication) must be recorded.

### Treatment Cycles (1-4)

Subjects will continue to record neuroma foot pain and use of rescue medication by IWRS/IVRS System at home throughout each Treatment Cycle.

### Treatment Visit 1/Treatment Day 1

### Pre-Injection Assessments

The following procedures will be performed pre-dose on Treatment Day 1 of each Treatment Cycle:
1. Complete physical examination (excluding a genitourinary exam) including weight.
2. Collection of blood for PK analysis (PK consented population only).
3. Clinical laboratory tests: chemistry, hematology, urinalysis.
4. Urine drug screen.
5. Urine pregnancy test.
6. Vital signs.
7. Sensory and motor examination of both feet.
8. Review IWRS/IVRS System entries and compliance with subject, and instruct subject to continue weekly entries (NPRS scores and use of rescue medication). Conduct subject retraining if non-compliant.
9. Neuroma foot pain rating at study visit (average walking pain and worst pain over last 24 hours) using NPRS.
10. Procedure pain (Baseline, pre-dose): Subjects will rate their current pain for the affected foot (NPRS; 0-10) at rest.
11. Foot function assessment.
12. QoL assessment.
13. Adverse events.
14. Concomitant medications and therapies. During the study, all medications and non-drug therapies (including rescue medication) be recorded.

### Treatment Day 1 Injection and Post-Injection Assessments

trans-Capsaicin injection will be performed using ultrasound-guided needle placement, with use of adjunct cooling. The following procedures should be performed for each injection:
i. Injection related pain will not be categorized as an adverse event, as pain post injection is assessed several times post injection.
ii. Adjunct use of cooling will be applied for 15 minutes prior to 1% lidocaine injection.
iii. Cooling device is removed for lidocaine injection, immediately followed by reapplying the cooling device for 10 minutes.
   Subject will rate his/her current pain at rest 10 minutes (±2 minutes) after lidocaine injection
iv. Replace adjunct cooling for 20 minutes.
v. At 30 minutes after lidocaine administration, remove the cooling device.
vi. Inject trans-capsaicin into the area of the affected foot's neuroma.
vii. Immediately after trans-capsaicin injection apply cooling (for a minimum of 30 minutes and up to 1 hour).

The following procedures will be performed post-injection on Treatment Day 1 of each Treatment Cycle. Note that injection related pain will not be captured as adverse events, as pain post injection is assessed several times post injection.
1. Subject will rate his/her current pain at rest 30 minutes (±5 minutes) after trans-capsaicin injection.
2. Adjunct cooling should be removed to assess pain and reapplied immediately after assessment of pain is recorded.
3. At 1 hour post trans-capsaicin injection:
   If adjunct cooling is still being used, cooling should be removed for assessment, adjunct cooling should no longer be used after 1 hour post trans-capsaicin injection.
   Subject will rate his/her current pain at rest 1 hour after trans-capsaicin injection (±10 minutes).
   Injection site assessment (erythema, edema): at 1 hour post-injection. Evaluated separately by the investigator or a trained designee using a categorical scale of “none, mild, moderate or severe”. Significant bruising or other clinically significant injection site reactions (other than erythema and edema) must be recorded as AEs.
4. At 2 hours post trans-capsaicin injection:
   Subject will rate his/her current pain at rest 2 hours after trans-capsaicin injection (±10 minutes).
   Injection site assessment (erythema, edema): at 2 hour post-injection.
5. Collection of blood at 0.25, 0.5, 1, 1.5, 2, 4, 8, 10, and 12 h post-dose, for PK analysis (PK consented population only) and first treatment cycle only; if any subjects in the PK population receive further treatment with trans-capsaicin, blood samples will be drawn pre-dose and at 2 h post-dose for calculation of the trans-capsaicin plasma concentrations.
6. Vital signs will be collected at discharge (approximately 2 hours post-injection, or 12 hours post-injection for the PK population).
7. When leaving the clinic, subjects should be instructed not to take a warm or hot bath or shower or expose the injected foot to heat within 24 hours after the injection.

### Treatment Visit 2/Week 1, Telephone Call

The study staff will telephone the subject at Week 1 (Visit 2) for the following assessments:
1. Adverse events.
2. Concomitant medications and therapies. Use of all medications and non-drug therapies (including rescue medication) must be recorded.

### Treatment Visit 3/Week 2, Site Visit

Subjects will return to the clinic at Visit 3 (Week 2) for the following assessments:
1. Vital signs.
2. Sensory and motor examination of both feet.
3. Injection site assessment (erythema, edema).
4. Review IWRS/IVRS System entries and compliance with subject, and instruct subject to continue weekly entries (NPRS scores and use of rescue medication). Conduct subject retraining if non-compliant.
5. Neuroma foot pain at study visit (average walking pain and worst pain over last 24 hours) using NPRS.
6. Neuroma foot pain: PGIC from the subject's most recent assessment.
7. Foot function assessment.
8. QoL assessment.
9. Adverse events.
10. Concomitant medications and therapies. Use of all medications and non-drug therapies (including rescue medication) must be recorded.

### Treatment Visit 4 (Treatment Cycles 1-4, Week 4, Site Visit)

Subjects will return to the clinic at Visit 4 (Week 4) for the following assessments:
1. Vital signs.
2. Sensory and motor examination of both feet.
3. Injection site assessment (erythema, edema).
4. Review IWRS/IVRS System entries and compliance with subject, and instruct subject to continue weekly entries (NPRS scores and use of rescue medication). Conduct subject retraining if non-compliant.
5. Neuroma foot pain at study visit (average walking pain and worst pain over last 24 hours) using NPRS.
6. Neuroma foot pain: PGIC from the subject's most recent injection.
7. Foot function assessment.
8. QoL assessment.
9. Adverse events.
10. Concomitant medications and therapies. Use of all medications and non-drug therapies (including rescue medication) must be recorded.

### Final Visit (Week 52) or Early Termination Visit

At Week 52 or upon early termination, subjects will return to the clinic for the following assessments:
1. Complete physical examination (excluding a genitourinary exam) including weight.
2. 12-lead ECG
3. Clinical laboratory tests: chemistry, hematology, urinalysis.
4. Urine drug screen.
5. Urine pregnancy test for females of childbearing potential.
6. Vital signs.
7. Sensory and motor examination. Assessed for both feet.
8. Neuroma foot pain rating at study visit (average walking pain and worst pain over last 24 hours) using NPRS.
9. Neuroma foot pain: PGIC from the subject's most recent assessment.
10. Foot function assessment.
11. QoL assessment.
12. Adverse events.
13. Concomitant medications and therapies. During the study, use of all medications and non-drug therapies (including rescue medication) must be recorded.

A subject who receives their last dose at Week 48 will complete both the Week 4 Treatment Cycle assessments and all additional Final Visit assessments at the same visit.

### Assessment of Pain Relief

The following tests are to be used in evaluating relief from pain due to the intermetatarsal neuroma:

### Average Walking and Worst Neuroma Foot Pain

Subjects will use an IWRS/IVRS System at bedtime to record on a weekly basis their average foot pain score with walking during the previous 24 hours. Neuroma foot pain with walking will be evaluated using a 0 to 10 NPRS (0=“no pain” and 10=“worst possible pain”). Subjects will also record their worst neuroma foot pain over the previous 24 hours using the NPRS.

### Neuroma Foot Pain Assessed at Study Visits

Subjects will rate their average neuroma foot pain score with walking during the previous 24 hours at each study visit. Neuroma foot pain will be evaluated using the NPRS. Subjects will also record their worst neuroma foot pain over the previous 24 hours using the NPRS.

### Foot Function Assessments

To evaluate any functional changes, at scheduled in-clinic study visits, subjects will complete the FFI-R.

### Patient Global Impression of Change

Subjects will rate change in neuroma foot pain as compared to the most recent assessment in each treatment cycle using the PGIC at each scheduled in-clinic study visit, according to the Schedule of Events.

### Need for Oral Rescue Medication to Treat Morton's Neuroma Pain

Subjects may only take oral OTC pain medications or prescription medication such as celecoxib (up to 200 mg twice daily) etc., as rescue medication for their neuroma foot pain. The number of days that the subject used rescue medication in the previous week will be recorded weekly by the subject in the IWRS/IVRS System. Additional rescue medication details will be collected at study visits and follow-up telephone calls in the source documents and eCRF, recorded as concomitant medications.

### Quality of Life

Quality of life will be assessed using a EQ-5D-5L scale at scheduled in-clinic study visits.

### Example 2—Administration of Two Doses of Capsaicin to Achieve Long Duration Relief from Pain Associated with an Intermetatarsal Neuroma

Twenty-seven adult, human patients experiencing pain due to an intermetatarsal neuroma were treated by administering a first dose of trans-capsaicin (200 μg of trans-capsaicin) and then, after at least 11 weeks, administered a second dose of trans-capsaicin (200 μg of trans-capsaicin). Patients rated their average walking pain due to the intermetatarsal neuroma on a Numeric Pain Rating Scale (NPRS), where pain is characterized by the patient on a scale of zero to ten (with zero being “no pain”, and ten being “worst possible pain”). Patients rated their average walking pain due to the intermetatarsal neuroma on (i) just prior to receiving the injection of trans-capsaicin and (ii) four (4) weeks after receiving each injection of trans-capsaicin. Patients reported a reduction in average walking pain due to the intermetatarsal neuroma when measured at four weeks after injection of trans-capsaicin for each administration of trans-capsaicin. Further description of experimental procedures and results are provided below.

### Part I—Experimental Procedures

trans-Capsaicin was administered to twenty-seven (27) adult, human patients experiencing pain due to an intermetatarsal neuroma according to the procedures described below. Prior to administering the first dose of trans-capsaicin in this study, patients reported an average walking pain due to the intermetatarsal neuroma of at least four on the Numeric Pain Rating Scale (NPRS). Patients received two doses of trans-capsaicin.

Administration of trans-Capsaicin

trans-Capsaicin was injected in the amount of 200 μg per dose by ultrasound-guided needle placement into the area of the neuroma (but not inserting the needle into the intermetatarsal neuroma itself). The dose of trans-capsaicin was injected as a 2 mL solution containing trans-capsaicin at a concentration of 100 μg/mL. Local anesthesia was performed with up to 4 mL of 1% lidocaine (without epinephrine) injected adjacent to the neuroma 30 minutes prior to injection of trans-capsaicin. Adjunct use of cooling was applied before 1% lidocaine injection; after lidocaine injection cooling was put back on for 30 minutes prior to trans-capsaicin injection. Cooling was removed for trans-capsaicin injection and then reapplied immediately following the injection.

trans-Capsaicin was supplied as a 2 mg/mL solution in PEG-300 (poly ethylene glycol having a number-average molecular weight of approximately 300 g/mol) and was diluted prior to injection with sterile water such that the final solution for injection contained 30% PEG-300 at a final concentration of 100 μg/mL trans-capsaicin.

The second dose of trans-capsaicin was administered to patients at a time ranging from 83 days to 196 days after administration of the first dose of trans-capsaicin in this study. The mean time period between administration of the first dose of trans-capsaicin and the second dose of trans-capsaicin in this study was 116 days.

### Evaluation of Pain Due to the Intermetatarsal Neuroma

Pain due to the intermetatarsal neuroma was evaluated by having patients rate their average walking pain due to the intermetatarsal neuroma on a Numeric Pain Rating Scale (NPRS), where pain is characterized by the patient on a scale of zero to ten (with zero being “no pain”, and ten being “worst possible pain”). Patients rated their average walking pain due to the intermetatarsal neuroma on (i) just prior to receiving the injection of trans-capsaicin and (ii) four (4) weeks after receiving each injection of trans-capsaicin.

### Part II—Results

There was a 1.6 point reduction in patients' reported average walking pain due to the intermetatarsal neuroma measured at four weeks after injection of the first dose of trans-capsaicin compared to patients' reported average walking pain prior to receiving the first dose of trans-capsaicin. There was a 2.3 point reduction in patients' reported average walking pain due to the intermetatarsal neuroma measured at four weeks after injection of the second dose of trans-capsaicin compared to patients' reported average walking pain just prior to receiving the second dose of trans-capsaicin. The results show that repeat injection of trans-capsaicin is effective in ameliorating pain due to an intermetatarsal neuroma.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of ameliorating pain for a duration of at least 6 months due to an intermetatarsal neuroma in a patient, comprising administering by injection into the patient's intermetatarsal space having an intermetatarsal neuroma at least a first dose of capsaicin and a second dose of capsaicin to ameliorate pain due to the intermetatarsal neuroma for a duration of at least 6 months, wherein the method is characterized by:
a. the first dose of capsaicin is in an amount ranging from about 150 μg to about 250 μg of capsaicin;
b. the second dose of capsaicin is in an amount ranging from about 150 μg to about 250 μg of capsaicin;
c. the second dose of capsaicin is administered no sooner than 3 months after administration of the first dose of capsaicin; and
d. if any additional dose of capsaicin is administered by injection into the patient's intermetatarsal space having an intermetatarsal neuroma, any such additional dose is in an amount ranging from about 150 μg to about 250 μg of capsaicin and any said additional dose is administered no sooner than 3 months after administration of the prior dose of capsaicin administered by injection into the patient's intermetatarsal space having an intermetatarsal neuroma.

2. The method of claim 1, wherein the first dose of capsaicin is in an amount ranging from about 175 μg to about 225 μg of capsaicin.

3. The method of claim 1, wherein the first dose of capsaicin is about 200 μg of capsaicin.

4. The method of claim 2, wherein the second dose of capsaicin is in an amount ranging from about 175 μg to about 225 μg of capsaicin.

5. The method of claim 2, wherein the second dose of capsaicin is about 200 μg of capsaicin.

6. The method of claim 4, wherein the any additional dose of capsaicin is in an amount ranging from about 175 μg to about 225 μg of capsaicin.

7. (canceled)

8. A method of ameliorating pain for a duration of at least 3 months due to an intermetatarsal neuroma in a patient, comprising administering by injection into the patient's intermetatarsal space having an intermetatarsal neuroma at least a first dose of capsaicin and a second dose of capsaicin to ameliorate pain due to the intermetatarsal neuroma for a duration of at least 3 months, wherein the method is characterized by:
a. the first dose of capsaicin is in an amount ranging from about 100 μg to about 1,000 μg of capsaicin;
b. the second dose of capsaicin is in an amount ranging from about 100 μg to about 1,000 μg of capsaicin;
c. the second dose of capsaicin is administered no sooner than 1 month after administration of the first dose of capsaicin; and
d. if any additional dose of capsaicin is administered by injection into the patient's intermetatarsal space having an intermetatarsal neuroma, any such additional dose is in an amount ranging from about 100 μg to about 1,000 μg of capsaicin and any said additional dose is administered no sooner than 1 month after administration of the prior dose of capsaicin administered by injection into the patient's intermetatarsal space having an intermetatarsal neuroma.

9-23. (canceled)

24. The method of claim 1, wherein the second dose of capsaicin is administered no sooner than 4 months after administration of the first dose of capsaicin.

25-40. (canceled)

41. The method of claim 24, wherein any additional dose of capsaicin subsequent to the second dose of capsaicin is administered at a time that is about 5 months after administration of the prior dose of capsaicin.

42-47. (canceled)

48. The method of claim 1, wherein over a duration of 1 year, the patient receives no more than three doses of capsaicin by injection into the patient's intermetatarsal space having an intermetatarsal neuroma.

49-54. (canceled)

55. The method of claim 48, wherein the pain is ameliorated for a duration of at least 12 months.

56-64. (canceled)

65. The method of claim 1, wherein any dose of capsaicin is injected into tissue adjacent to the intermetatarsal neuroma, whereby the medical instrument performing the injection does not penetrate into the intermetatarsal neuroma.

66. (canceled)

67. The method of claim 1, further comprising cooling tissue adjacent to the intermetatarsal neuroma before administering capsaicin.

68. The method of claim 1, further comprising cooling tissue adjacent to the intermetatarsal neuroma after administering capsaicin.

69. The method of claim 1, further comprising administering a local anesthetic agent to the patient immediately prior to injecting the capsaicin in order to ameliorate any pain experienced by the patient due to administering the capsaicin.

70-73. (canceled)

74. The method of claim 1, wherein any second dose or additional dose of capsaicin may be administered to the patient without administering a local anesthetic agent to the patient immediately prior to injecting the capsaicin, and any pain experienced by the patient due to the administration of a second dose or additional dose of capsaicin is no greater than a score of mild on the Injection Pain Scale.

75-77. (canceled)

78. The method of claim 1, wherein the patient experiences pain due to the intermetatarsal neuroma of at least a level 4 at some point during the twenty-four hour period prior to administering the first dose of capsaicin.

79-93. (canceled)

94. The method of claim 1, wherein the method is characterized by achieving a reduction in average walking foot pain due to the intermetatarsal neuroma by at least 2 on the Numeric Pain Rating Scale (NPRS) for a duration of at least 3 months.

95-96. (canceled)

97. The method of claim 1, wherein the method is characterized by achieving a reduction in average walking foot pain due to the intermetatarsal neuroma by at least 2 on the Numeric Pain Rating Scale (NPRS) for a duration of at least 6 months.

98-106. (canceled)

107. The method of claim 1, wherein the method is characterized by reducing the patient's average walking foot pain due to the intermetatarsal neuroma so that the patient's average walking foot pain due to the intermetatarsal neuroma is no greater than 1 on the Numeric Pain Rating Scale (NPRS) for a duration of at least 6 months

108-117. (canceled)

118. The method of claim 1, wherein the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 3 months.

119-120. (canceled)

121. The method of claim 1, wherein the method is characterized by reducing the patient's worst neuroma foot pain due to the intermetatarsal neuroma so that the patient's worst neuroma foot pain due to the intermetatarsal neuroma is no greater than 1 on the Numeric Pain Rating Scale (NPRS) for a duration of at least 6 months.

122-131. (canceled)

132. The method of claim 1, wherein upon administration of a first dose of capsaicin, the patient experiences an improvement in their Revised Foot Function Index (FFI-R) score of at least 1 within 2 weeks after administration of the dose of capsaicin and lasting for a duration of at least 2 months.

133-139. (canceled)

140. The method of claim 1 wherein during the 24 hour period prior to administration of the first dose of capsaicin, the patient suffers from one or more of the following:
a. an average walking foot pain due to the intermetatarsal neuroma of at least 4 on the Numeric Pain Rating Scale (NPRS);
b. a worst neuroma foot pain due to the intermetatarsal neuroma of at least 4 on the Numeric Pain Rating Scale (NPRS); or
c. a Revised Foot Function Index (FFI-R) score indicating the patient experiences at least two of the following: (i) moderate pain due to the intermetatarsal neuroma, (ii) moderate stiffness due to the intermetatarsal neuroma, and (iii) moderate difficulty in a physical activity due to the intermetatarsal neuroma.

141-144. (canceled)

145. The method of claim 1, wherein the patient is an adult human male, or an adult human female.

146. (canceled)
